(19) United States
(12) Patent Application Publication
(10) Pub. No.: US 2010/0222767 A1
(43) Pub. Date: Sep. 2, 2010

(54) Markers for needles including temporary and pen injector types

(76) Inventor: Peter Jon Gluck, Mission Viejo, CA (US)

Correspondence Address: PJ Gluck, Esq., 26892 Preciados Drive, Mission Viejo, CA, 92691, US

(21) Appl. No.: 12/653,376

(22) Filed: Dec. 12, 2009

Related U.S. Application Data
(60) Provisional application No. 61/206,714, filed on Feb. 2, 2009.

(51) Int. Cl. A61M 5/32

(52) U.S. Cl. 604/506; 604/272

(57) ABSTRACT

A Marker on the dorsal (top) side of a needle allows a visually challenged user to maintain an orientation of the needle from a point within the user's comfortable visual focusing range to a desired injection site

### CROSS REFERENCE TO RELATED APPLICATIONS

This application claims the Paris Convention Priority, and all legal benefits of U.S. Ser. No. 61/206714 (Feb. 2, 2009), which is likewise incorporated by reference.

### BACKGROUND OF THE DISCLOSURE

The present disclosure relates to improved processes and products which facilitate injections, particularly by users themselves, of medicaments, drugs and other injectables, particularly with temporary needles and “pen-type” injectors.

### OBJECT AND SUMMARY OF THE DISCLOSURE

Briefly stated, a marker on the dorsal (top) side of a needle allows a visually challenged user to maintain an orientation of the needle from a point within the user's comfortable visual focusing range to a desired injection site. According to embodiments there is provided a needle marking system, comprising, in combination: an effective amount of marking material to allow a user to differentiate a dorsal side from a ventral side of a needle when the needle is viewed proximal to an injection site by a user; whereby, when the marking material is applied to a subject needle, a user can note a desired spatial orientation both at a comfortable focusing range, and beyond a comfortable focusing range because the marking material allows the user to align a desired aspect of the needle's tip with the dorsal and ventral side orientated such that an injection may be made optimizing the interface angle.

### BRIEF DESCRIPTION OF THE FIGURES

FIG. 1 shows a schematized needle with the present disclosure thereopun;

FIG. 2 shows a side view of the same.

### DETAILED DESCRIPTION OF THE DISCLOSURE

The present inventor has discovered that temporary needles, for example, those used by Type II diabetics, as “pen-injector” system components cannot be visualized (particularly for those with presbyopia, or other visual disorders that come with age, diabetes, or lack of accomodative ability) equally at an injection site (eg. the gut) and proximal to the eye. In other words, needles need to enter, for example, the skin with an orientation whereby the top-side is aligned such that the everting tip enters the skin at the proper angle, so when one adjusts the orientation and then attempts to move to the site and inject, it is not possible to confirm the orientation is the same. This means that often the injection is ‘botched’ when the angle changes, ripping or tearing skin or causing other issues, infection, etc.

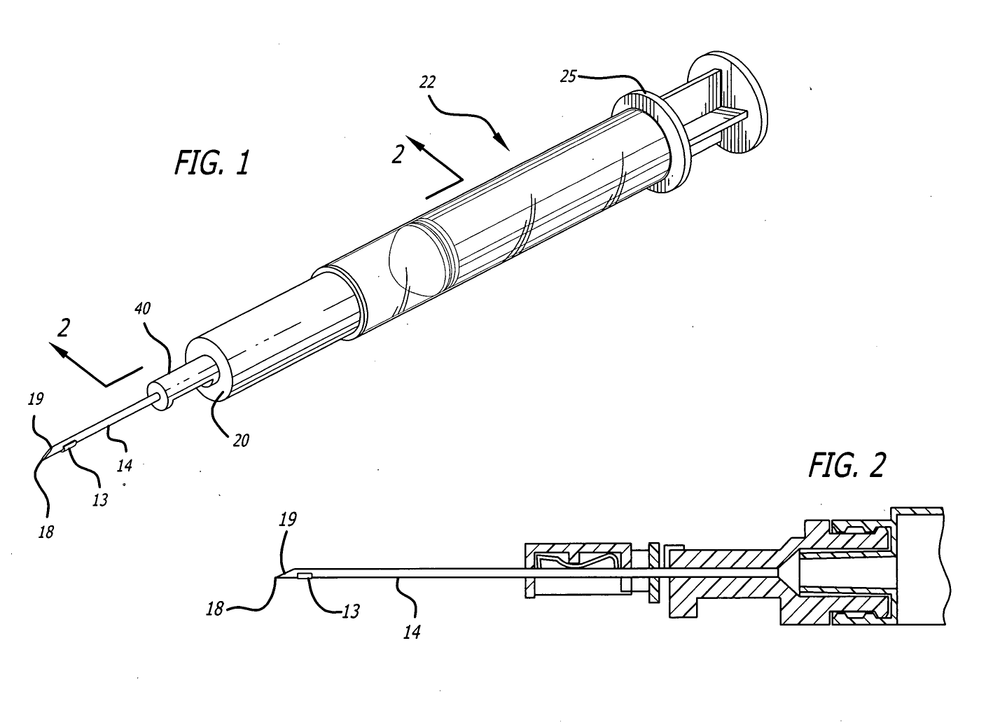

Referring now to FIG. 1, a a schematized injector system (eg. the BYETTA® brand of injectable EXANATIDE) is shown at 22, with plunger mechanism 26, used to push drugs through for example a temporary needle Novofine 30 Disp Ned 8 mm 100s 30G ⅓ (Novo Nordisk) 14, but tip 18, and everted aspect 19 must be maintained orthogonal to the injection site to optimize subcutaneous drug delivery. Present invention 13 can be seen equally at the injection site (even if patient is visually challenged), so that mediciation passes through barrel 40 when it leaves the distal end of injector 20.

Sight guide 2, shows that in at least two planes, alignment of marker 13, on dorsal surface of needle 14 allows a user to move the needle from the line of sight of user (eg. proximal to a user's eyes, where even elderly and sight-challenged users can align everted aspect 19) to the injection site, without losing the spatial orientation as shown by sight guide 2.

FIG. 2 likewise shows a side view of marker 13 relative to tip 18, and everted portion 19, of needle 14.

Any patents, publications, or other references mentioned in this application for patent are hereby incorporated by reference. In addition, as to each term used it should be understood that unless its utilization in this application is inconsistent with such interpretation, common dictionary definitions should be understood as incorporated for each term and all definitions, alternative terms, and synonyms such as contained in at least one of a standard technical dictionary recognized by artisans and the Random House Webster's Unabridged Dictionary, latest edition are hereby incorporated by reference.

Finally, all referenced listed in the Information Disclosure Statement or other information statement filed with the application are hereby appended and hereby incorporated by reference; however, as to each of the above, to the extent that such information or statements incorporated by reference might be considered inconsistent with the patenting of this/these invention(s), such statements are expressly not to be considered as made by the applicant(s).

In this regard it should be understood that for practical reasons and so as to avoid adding potentially hundreds of claims, the applicant has presented claims with initial dependencies only.

Support should be understood to exist to the degree required under new matter laws—including but not limited to United States Patent Law 35 USC 132 or other such laws—to permit the addition of any of the various dependencies or other elements presented under one independent claim or concept as dependencies or elements under any other independent claim or concept.

To the extent that insubstantial substitutes are made, to the extent that the applicant did not in fact draft any claim so as to literally encompass any particular embodiment, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative embodiments.

Further, the use of the transitional phrase “comprising” is used to maintain the “open-end” claims herein, according to traditional claim interpretation. Thus, unless the context requires otherwise, it should be understood that the term “compromise” or variations such as “comprises” or “comprising”, are intended to imply the inclusion of a stated element or step or group of elements or steps but not the exclusion of any other element or step or group of elements or steps.

Such terms should be interpreted in their most expansive forms so as to afford the applicant the broadest coverage legally permissible.

While the method and agent have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

It should also be understood that a variety of changes may be made without departing from the essence of the invention. Such changes are also implicitly included in the description. They still fall within the scope of this invention. It should be understood that this disclosure is intended to yield a patent covering numerous aspects of the invention both independently and as an overall system and in both method and apparatus modes.

Further, each of the various elements of the invention and claims may also be achieved in a variety of manners. This disclosure should be understood to encompass each such variation, be it a variation of an embodiment of any apparatus embodiment, a method or process embodiment, or even merely a variation of any element of these.

Particularly, it should be understood that as the disclosure relates to elements of the invention, the words for each element may be expressed by equivalent apparatus terms or method terms—even if only the function or result is the same.

Such equivalent, broader, or even more generic terms should be considered to be encompassed in the description of each element or action. Such terms can be substituted where desired to make explicit the implicitly broad coverage to which this invention is entitled.

It should be understood that all actions may be expressed as a means for taking that action or as an element which causes that action.

Similarly, each physical element disclosed should be understood to encompass a disclosure of the action which that physical element facilitates.

Any patents, publications, or other references mentioned in this application for patent are hereby incorporated by reference. In addition, as to each term used it should be understood that unless its utilization in this application is inconsistent with such interpretation, common dictionary definitions should be understood as incorporated for each term and all definitions, alternative terms, and synonyms such as contained in at least one of a standard technical dictionary recognized by artisans and the Random House Webster's Unabridged Dictionary, latest edition are hereby incorporated by reference.

Finally, all referenced listed in the Information Disclosure Statement or other information statement filed with the application are hereby appended and hereby incorporated by reference; however, as to each of the above, to the extent that such information or statements incorporated by reference might be considered inconsistent with the patenting of this/these invention(s), such statements are expressly not to be considered as made by the applicant(s).

In this regard it should be understood that for practical reasons and so as to avoid adding potentially hundreds of claims, the applicant has presented claims with initial dependencies only.

Support should be understood to exist to the degree required under new matter laws—including but not limited to United States Patent Law 35 USC 132 or other such laws—to permit the addition of any of the various dependencies or other elements presented under one independent claim or concept as dependencies or elements under any other independent claim or concept.

To the extent that insubstantial substitutes are made, to the extent that the applicant did not in fact draft any claim so as to literally encompass any particular embodiment, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative embodiments.

Further, the use of the transitional phrase “comprising” is used to maintain the “open-end” claims herein, according to traditional claim interpretation. Thus, unless the context requires otherwise, it should be understood that the term “compromise” or variations such as “comprises” or “comprising”, are intended to imply the inclusion of a stated element or step or group of elements or steps but not the exclusion of any other element or step or group of elements or steps.

Such terms should be interpreted in their most expansive forms so as to afford the applicant the broadest coverage legally permissible.

1. A needle marking system, comprising, in combination:
an effective amount of marking material to allow a user to differentiate a dorsal side from a ventral side of a needle when the needle is viewed proximal to an injection site by a user;
whereby, when the marking material is applied to a subject needle, a user can note a desired spatial orientation both at a comfortable focusing range, and beyond a comfortable focusing range because the marking material allows the user to align a desired aspect of the needle's tip with the dorsal and ventral side orientated such that an injection may be made optimizing the interface angle.

2. A temporary needle marker, comprising, in combination:
an effective amount of at least one of a color, a visually perceptible indicator, and other means for showing a user at least the top side of a needle;
wherein when a user selects a desired orientation of the temporary needle said orientation can be maintained, at a location distal from the user's ideal visual range for perceiving the orientation because the at least one of a color, a visually perceptible indicator, and other means for showing a user at least the top side of a needle can be observed, ensuring that the angle of the top side of the needle relative to the desired injection ingress site can be maintained.

3. A process for creating a needle marking device, comprising, in combination:
applying an effective amount of at least one of a color, a visually perceptible indicator, and other means for showing a user at least the top side of a needle; and
packaging the same.

4. Products, by the process of claim 3.

5. A method for delivering an injection through the skin, comprising:
aligning a temporary needle such that the dorsal surface of the needle and everted tip portion are attached to a syringe-type of injection or injector pen;
confirming that the dorsal surface of the needle and everted tip portion attached to a syringe-type of injection or injector pen remains orthogonal to a desired injection ingress site by visually aligning a marker on the temporary needle; and
performing the injection.
